(19) United States
(12) Patent Application Publication
(10) Pub. No.: US 2023/0260592 A1
(43) Pub. Date: Aug. 17, 2023

(54) Method and Device for Protein-Protein Docking Based on Identification of Low-Entropy Hydration Layer on Protein Surface

(71) Applicant: Harbin Institute of Technology, Harbin (CN)

(72) Inventors: Lin Yang, Harbin (CN); Chengyu Hou, Harbin (CN); Chenchen Liao, Harbin (CN); Xiaodong He, Harbin (CN)

(73) Assignee: Harbin Institute of Technology, Harbin (CN)

(21) Appl. No.: 17/984,660

(22) Filed: Nov. 10, 2022

(30) Foreign Application Priority Data
Feb. 15, 2022 (CN) 202210138581.X

(51) Int. Cl. G16B 15/30; G16B 15/20

(52) U.S. Cl. CPC G16B 15/30; G16B 15/20

(57) ABSTRACT

Provided is a method and a device for protein-protein docking based on identification of a low-entropy hydration layer on a protein surface. Hydrophobic groups on a protein surface and hydrophobic groups containing a small amount of oxygen and nitrogen atoms, as well as some hydrophilic groups in intramolecular hydrogen bonds formed on the protein surface are identified as low-entropy areas. In a computer program, according to a low-entropy hydration layer theory on the protein surface, some nitrogen and oxygen hydrophilic atoms of the protein are changed into hydrophobic carbon atoms; the protein surface is cut into multiple planes, atoms in a hydrophobic connection region are selected in each plane, an area and a shape of surface atoms are calculated in each hydrophobic connection region, and a plane is selected with a maximum hydrophobic connection area; a protein-protein docking site is predicted using the connection region as a possible docking site.

### CROSS REFERENCE TO RELATED APPLICATION

This patent application claims the benefit and priority of Chinese Patent Application No. 202210138581.X, filed with the China National Intellectual Property Administration on Feb. 15, 2022, the disclosure of which is incorporated by reference herein in its entirety as part of the present application.

### TECHNICAL FIELD

The present disclosure relates to a method and a device for predicting a protein-protein docking structure.

### BACKGROUND

Proteins and their products are the basis of life on the Earth and are involved in almost all known biochemical reactions and phenomena of life. There are trillions of different proteins in Earth organisms, with the biological function and activity of each protein expressed through a unique three-dimensional shape. A process by which proteins form natural three-dimensional structures is called protein folding, with its mechanism and laws as a basis of molecular biology, biophysics, and biochemistry. Protein folding is thought to be primarily guided by a variety of physical forces: (i) hydrogen bond formation, (ii) Van der Waals’ forces, (iii) electrostatic forces, (iv) hydrophobic interactions, (v) entropy, and (vi) temperature. Protein folding realizes the functionalization of polypeptide chains, and interpretation and prediction of the protein folding is of great significance to biology, pathology, genetics, and pharmacology.

The tertiary structure of proteins is formed by the folding of polypeptide chains. A process of the tertiary structures being assembled into a quaternary structure as subunits is essentially the same as that of protein docking. In the quaternary structure, the subunits are mainly combined by hydrophobic interactions, followed by hydrogen bonds and an extremely small amount of ionic bonds. The hydrogen bonding between subunit structures generally occurs between the hydrophilic groups on a surface of the subunit. The formation of these hydrogen bonds also requires the hydrophilic side chains on a surface of the subunit structure to get rid of the hydrogen bonding of environmental water molecules. The hydrophilicity of a residue side chain is generally expressed only by C—O or N—H groups at a top of the side chain. According to enthalpy calculation, the hydrogen bonding between the C—O group and the N—H group on the side chain between the subunits may also lead to an increase in the enthalpy of a system. Therefore, hydrophilic groups on the surface of the subunit structure cannot spontaneously get rid of the hydrogen bonding of surface water molecules, such that the formation of hydrogen bonds between subunits also requires enthalpy-entropy compensation. For the tertiary structure as a subunit, its surface is generally distributed with localized hydrophobic regions. During the formation of a quaternary structure of proteins, a local hydrophobic collapse between subunits at a docking interface can provide a source of the enthalpy-entropy compensation and promote the formation of hydrogen bonds between subunits, thereby forming a precise quaternary structure. Therefore, the assembly of subunits into a quaternary structure is driven by hydrophobic interactions and enthalpy-entropy compensation mechanisms.

It must be pointed out that the protein surface has a hydration layer with a thickness of about 1 nm to 2 nm, and it is found that the dynamic behavior of water molecules in the hydration layer is significantly different from that of free water molecules. The experimental data published by Dongping Zhong in Proceedings of the National Academy of Sciences (PNAS) shows that the water molecules in the hydration layer on the protein surface have a movement speed of only one percent of that of the free water molecules, and the water molecules in the hydration layer has lower entropy. The long-range hydrophobic interactions between protein molecules and a resulting entropy increase should be a core driving force for protein-protein docking. Due to the existence of hydration layers, the enthalpy of a system may increase due to hydrogen bonding and electrostatic interactions between protein molecules, such that the protein-protein docking is driven by enthalpy-entropy compensation.

The mechanism of protein-protein interaction and recognition is an important topic in biology, pathology, genetics, and pharmacology. The sites of protein-protein interactions represent important viral infection and immune mechanisms. Taking the current global pandemic COVID-19 as an example, the root cause lies in the specific and high-affinity binding between a spike protein (S protein) of the COVID-19 virus and a human ACE2 protein. That is to say, the “protein-protein docking” is a core problem of researches on virus infection mechanism.

In an early stage of the epidemic, due to the lack of effective methods to predict a molecular structure and binding energy of a binding state of the virus and the receptor, experimental methods generally can only quickly decipher gene sequence information of the virus, while the gene sequence information cannot be used to directly decipher important information such as virus infectivity and probability of vaccine breakthrough infection. Therefore, it is an inevitable trend of technological development in this field to predict a complex structure of the protein-protein docking by computational methods. However, the currently popular computational methods for protein-protein docking assist in predicting the structure of protein complexes using a scoring function selected from natural conformation, while there are still relatively few studies on a physical mechanism of the protein-protein docking based on “enthalpy-entropy compensation” to solve this important scientific problem.

In view of the above research background, the present disclosure proposes a method for protein-protein docking based on identification of a low-entropy hydration layer on a protein surface. Different from traditional protein-protein docking prediction algorithms based on noncovalent interactions (such as electrostatic interactions, Van der Waals’ forces, hydrogen bonds, and ionic bonds), the method can find a matching relationship of the hydrophobic interactions between proteins based on identifying a low-entropy region of the hydration layer on the protein surface, thereby accurately predicting a protein-protein docking structure. This novel method elucidates the essential drivers of protein recognition and docking, and resolves the principles of protein interactions, which is of great significance for understanding protein function and treating protein-related diseases. This method can provide a number of important virus infection mechanism information in the absence of clinical data, provide a scientific data support for the decision-making of accurate and effective national defense and epidemic prevention measures, and improve a rapid response capability of military and civilians to virus epidemic prevention and treatment. In addition, the method can be used to predict action sites of the proteins and drug molecules, and effectively evaluate and screen the interaction sites that are close to a real state. Therefore, the method can avoid blind, inefficient, and low-quality drug development processes, shorten a drug molecule development time, and save a lot of manpower and material resources.

### SUMMARY

In order to avoid inaccurate sites of current protein docking, the present disclosure proposes a new method for identifying a low-entropy region in a hydration layer on a protein surface. The hydration layers of certain hydrophilic groups surrounded by hydrophobic groups on the protein surface are identified as low-entropy regions, which are a result of surface tension and confinement. This new method for predicting a structure of a protein complex can rapidly and accurately predict a complex structure of protein-protein docking.

The present disclosure provides a method for protein-protein docking based on identification of a low-entropy hydration layer on a protein surface, including the following steps:
step 1, rubbing heavy atoms on a protein surface, where the heavy atoms include carbon atoms, nitrogen atoms, oxygen atoms, and sulfur atoms; calculating an average spatial coordinate of the heavy atoms in the protein, and dividing protein atoms into 20 regions with 20 vertices of a regular dodecahedron and using the average spatial coordinate as a center of projection;
step 2, for the protein in each obtained rubbing area, using a direction of the center of projection to the vertices of the regular dodecahedron as a z-axis, and determining surface atoms of the protein according to a maximum distance z from the center of projection to the protein atoms on the z-axis;
step 3, traversing the surface atoms, and changing a hydrophobicity and a hydrophilicity of the atoms according to amino acids and the surface atoms of the protein surface:
   case 1: if the amino acids of the protein surface are selected from the group consisting of leucine (Leu), tyrosine (Tyr), tryptophan (Trp), isoleucine (Ile), methionine (Met), phenylalanine (Phe), arginine (Arg), and lysine (Lys), and when carbonyl oxygen atoms or amide nitrogen atoms on backbone of the amino acids are exposed on the protein surface, changing the carbonyl oxygen atoms or the amide nitrogen atoms on the backbone to hydrophobic atoms, indicating that hydrophilic oxygen and nitrogen atoms of the backbone of residue Leu, Tyr, Trp, Ile, Met, Phe, Arg, or Lys become the hydrophobic atoms;
   case 2: if the amino acids of the protein surface belong to residue Trp, Tyr, Lys, and the Met, changing oxygen and nitrogen atoms of side chains of the amino acids to the hydrophobic atoms, indicating that the hydrophilic oxygen and nitrogen atoms at a head of the side chains of residue Trp, Tyr, Lys, and Met become the hydrophobic atoms; and
   case 3: if hydrogen bonds are formed between the oxygen and nitrogen atoms of the protein surface, whether in an “α-helix” and a “β-sheet” of a secondary structure of the protein or in elsewhere, changing the oxygen and nitrogen atoms that form the hydrogen bonds to the hydrophobic atoms;
step 4, after changing the hydrophobicity and the hydrophilicity of the atoms in the 20 regions of the protein in step 3, re-fitting a plane by a least square method to the surface atoms of the protein in each of the 20 regions, as a candidate protein docking plane; calculating an average distance di between a central coordinate position (xi, yi) of each of the protein docking planes in the 20 regions and all atoms on the plane, where i is a serial number of the rubbing area;
   after fitting 20 planes, comparing obtained planes and excluding duplicate planes; during excluding the duplicate planes, if two fitting planes have an included angle of less than or equal to 10° from a spatial origin to vertical vectors of the respective planes, regarding the two fitting planes as a same plane; and
   denoting remaining fitting planes as surface planes, and for each surface plane, selecting a central atom in a hydrophobic connection region, and marking atoms in the hydrophobic connection region;
step 5, calculating a hydrophobic area of the hydrophobic atoms on a surface of each hydrophobic connection region separately; and
step 6, selecting first three hydrophobic connection regions with a maximum hydrophobic area in the protein as possible docking positions, and docking two proteins to be docked.

Further, step 1 includes the following steps:
reading data information in a protein data bank (PDB) structure file of the protein, to obtain a three-dimensional spatial coordinate of each heavy atom on the protein surface; and
dividing the protein atoms into the 20 regions with the 20 vertices of the regular dodecahedron as follows: the regular dodecahedron has the 20 vertices, and the average spatial coordinate of all atoms of the protein is used as a spatial origin; if in a spatial area, an angle is less than 41° between a vector pointing to each atom from the spatial origin and a vector pointing to the vertex, the atom is divided into this spatial area; the 20 vertices of the regular dodecahedron divide the protein atoms into 20 areas, and one divided area of the protein surface is regarded as one rubbing area.

Further, in step 2, a process of determining the surface atoms of the protein according to the maximum distance z from the center of projection to the protein atoms on the z-axis specifically includes:

selecting externally lateral atoms on 30% of the maximum distance z from the center of projection to the protein atoms on the z-axis as the surface atoms of the protein, that is, selecting atoms with a distance from the center of projection of greater than 70% × d_{surface} as the surface atoms of the protein, where the d_{surface} is a distance from the center of projection to a maximum z-coordinate of the atoms of the protein surface on the z-axis.

Further, in step 4, a process of denoting the remaining fitting planes as the surface planes, and for each surface plane, selecting the central atom in the hydrophobic connection region specifically includes:
transforming atomic coordinates into a three-dimensional space with the surface plane as an xy plane by coordinate transformation; taking xy coordinates of the atomic coordinates, if in a circle centered on oxygen and nitrogen atoms and with a radius of 5 angstroms, there are no other oxygen and nitrogen atoms, not using this atom as a boundary in a subsequent search for the boundary; otherwise, in the xy plane, with oxygen and nitrogen atoms as the boundaries, assigning carbon and sulfur atoms as 1 in a circle centered on corresponding oxygen and nitrogen atoms and with a radius of 3 angstroms; taking assigned carbon and sulfur atoms as a center, if there are unassigned carbon and sulfur atoms in a circle centered on the assigned carbon and sulfur atoms and with a radius of 3 angstroms, adding up values of the assigned atoms in a circle centered on the unassigned carbon and sulfur atoms and with a radius of 3 angstroms as values of the unassigned carbon and sulfur atoms; at this time, only calculating the values of the unassigned carbon and sulfur atoms, and recording as pseudo-valuation of the unassigned carbon and sulfur atoms, while not assigning the unassigned carbon and sulfur atoms; when all atoms are searched for one round, assigning the pseudo-valuation of the unassigned carbon and sulfur atoms to the corresponding unassigned carbon and sulfur atoms, and starting a new round of assignment until all carbon and sulfur atoms complete the assignment; and
for each carbon and sulfur atom, in a circle centered on the carbon and sulfur atoms and with a radius of 3 angstroms, if values of the carbon and sulfur atoms are greater than or equal to values of surrounding atoms, regarding the carbon and sulfur atoms as central atoms of the corresponding hydrophobic connection region.

Further, in step 4, a process of marking atoms in the hydrophobic connection region specifically includes:

taking the central atom of the hydrophobic connection region as a center and 10° as a step size, dividing an area around the central atom, and selecting atoms with a value of greater than or equal to 3 in a fan-shaped area corresponding to each 10°; when an atom with a value of less than 3 appears for the first time, selecting a distance from an atom with a value of 3 closest to the atom with a value of less than 3 to the center as a cut-off distance; and selecting atoms within the cut-off distance as atoms within the hydrophobic connection region.

Further, in step 5, a process of calculating the hydrophobic area of the hydrophobic atoms on the surface of each hydrophobic connection region separately specifically includes:
step 5.1, for the surface heavy atoms of the protein in each hydrophobic connection region, displaying the surface heavy atoms as a sphere with an action radius of 1.8 angstroms; with each surface heavy atom as a center, making a hemisphere in a projection direction, where the hemisphere is a hemispherical shell;
step 5.2, establishing a two-dimensional grid with an interval of 0.1 angstrom on a plane of the hydrophobic connection region, and recording height information and a heavy atom type of the corresponding protein surface in each two-dimensional grid; and
step 5.3, establishing a surface with a radius of 1.8 angstroms as an action radius of the heavy atoms, with possibility of voids and discontinuities on the surface, where the hemispherical shells of the two heavy atoms have no intersection, and a distance between the two atoms is less than 6 angstroms, as shown in FIG. 3; taking a connection between a fan-shaped region formed by the hemispherical shell of one heavy atom with a projection direction in an included angle of 45° and a fan-shaped region formed by the hemispherical shell of another heavy atom with the projection direction in an included angle of 45° as an interpolation area; taking intersections of the connection and the two fan-shaped regions formed by the hemispherical shells of the two heavy atoms with the projection direction in an included angle of 45° as interpolation endpoints, obtaining a plane of a cavity between the hemispherical shells of the two heavy atoms by an interpolation, and abbreviating the plane of the cavity as an interpolation plane, as shown in FIG. 4; and
at the cavity, selecting a surface of the heavy atom at 45° to the projection direction as a surface connection point, and conducting cubic spline interpolation to obtain a type of the heavy atom and a three-dimensional height of the interpolation at the cavity, and then determining a space area corresponding to each grid in the two-dimensional grid, and then determining an area of the hydrophobic connection region.

Further, in step 6, a process of selecting the first three hydrophobic connection regions with a maximum hydrophobic area in the protein as the possible docking positions, and docking the two proteins to be docked specifically includes:
denoting the hydrophobic connection regions of the two proteins to be docked as a hydrophobic connection region A and a hydrophobic connection region B, respectively; determining a search range on the hydrophobic connection region A or the hydrophobic connection region B according to a relatively high average distance d=max(di, dj) corresponding to the two hydrophobic connection regions;
fixing the hydrophobic connection region A, establishing a 2d × 2d two-dimensional grid with an interval of 3 angstroms using a center coordinate of the hydrophobic connection region A as a surface origin of the hydrophobic connection region, and denoting area boundaries of the search range corresponding to the 2d × 2d of the hydrophobic connection region A as (x_max, y_max); locating a center coordinate of the hydrophobic connection region B in grid points of the two-dimensional grid in sequence, and rotating at an interval of 5° and calculating a docking situation of the two hydrophobic connection regions at each grid point; a calculation method includes the following steps:
   taking a normal vector of a fitting plane of each of the hydrophobic connection region A and the hydrophobic connection region B as a z-axis of the fitting plane, making the two hydrophobic connection regions approach on the z-axis of the fitting plane, placing the hydrophobic connection region B above the hydrophobic connection region A, and gradually reducing a height of the hydrophobic connection region B, that is, making the hydrophobic connection region B gradually approach the hydrophobic connection region A; where making the two hydrophobic connection regions gradually approach on the plane is to gradually overlap the z-axes of the two hydrophobic connection regions; however, if the z-axis of the hydrophobic connection region A faces upwards, the z-axis of the hydrophobic connection region B faces downwards, which is similar to conducting a coordinate transformation on the hydrophobic connection region B; and after the transformation, the two areas are in a same coordinate system;
   according to the process of the hydrophobic connection region B gradually approaching the hydrophobic connection region A, setting a minimum distance to be 1 angstrom between the atoms on the hydrophobic connection region A and the hydrophobic connection region B, and denoting a position of the minimum distance as a space docking position; in the space docking position, finding out minimum x- and y-axis coordinates of the atomic coordinates of the hydrophobic connection region A and the hydrophobic connection region B, denoting as (x_min, y_min), so as to facilitate the subsequent grid establishment and docking calculation;
   after the coordinate (x_min, y_min) are obtained, pressing the atoms in the hydrophobic connection regions A and B to an x-y plane, calculating atoms closest to the coordinate (x_min, y_min) in the hydrophobic connection regions A and B in the x-y plane, respectively, denoting two obtained atoms as an atom a and an atom b, taking an average of z-axis heights corresponding to respective spatial coordinates represented by the atom a and the atom b under the space docking position as a docking plane z value, and then determining a three-dimensional spatial coordinate (x_min, y_min, z); calculating a real distance from the atom a and the atom b in the space docking position separately using the coordinate (x_min, y_min, z), and if there is a distance of not less than 6 angstroms in the two distances, determining that the hydrophobic connection region A and the hydrophobic connection region B have no docking surface at the coordinate; otherwise, determining that there is a docking surface between two proteins at the coordinate;
   if there is no docking surface at the current space docking position, moving the hydrophobic connection region B on the two-dimensional grid of the hydrophobic connection region A, with a movement step size of (x, y) by 0.1 angstrom, that is: y increases by 0.1 once, and x goes from x_min to x_max; and y increases by 0.1 once more, and x goes from x_min to x_max once more; repeating the above step, with a range of y changing from y_min to y_max, until all coordinates are traversed;
   when there is a docking surface between the two proteins, recording a type of the docking surface under the space docking position;
   when there is a docking surface between the two proteins, determining a docking type at a docking interface of the hydrophobic connection regions A and B under the current space docking position, and adjusting the docking plane z value to a z-axis height corresponding to a same distance from the atom a and the atom b; where
   the docking type includes carbon atom-carbon atom, carbon atom-oxygen and nitrogen atoms, and oxygen and nitrogen atoms-oxygen and nitrogen atoms, and areas of different docking types at the docking interface of the hydrophobic connection regions A and B are calculated according to the docking type.

Further, in step 6, if there are multiple docking positions, a maximum complete docking area is selected as the docking position, and a complete docking area of the two proteins is integrally calculated at the position.

The present disclosure further provides a device for protein-protein docking based on identification of a low-entropy hydration layer on a protein surface, including a memory, where the memory stores at least one instruction, and the at least one instruction is loaded and executed by a processor to implement the method for protein-protein docking based on identification of a low-entropy hydration layer on a protein surface.

Further, the device further includes a processor, where the processor loads and executes the at least one instruction stored in the memory to implement the method for protein-protein docking based on identification of a low-entropy hydration layer on a protein surface.

### Beneficial Effects

In the present disclosure, protein docking sites are predicted based on a low-entropy hydration layer mechanism. Area maximization and shape matching of low-entropy regions at the protein docking sites are the most effective means to determine protein-protein interaction sites, and are the latest achievements in current protein docking theory. The low-entropy hydration layer mechanism can accurately predict an interface and sites of the protein-protein docking, which is a brand-new protein-protein docking theory, and can rapidly and accurately predict a protein-protein docking structure. In addition, the method can be used to predict action sites of the proteins and drug molecules, and effectively evaluate and screen the action sites that are close to a real state.

### BRIEF DESCRIPTION OF THE DRAWINGS

FIG. 1 shows a schematic diagram of a hemispherical shell projection direction (front view) of an atomic surface;

FIG. 2 shows a schematic diagram of a connection point on a hemispherical shell in an included angle of 45° with a projection direction in a three-dimensional space (top view);

FIG. 3 shows a schematic diagram of an interpolation between surfaces of two unconnected atoms;

FIG. 4 shows a schematic diagram of height information of an interpolation plane; and

FIG. 5 shows an effect of a low-entropy hydration layer at docking sites of a protein surface in the present disclosure.

### DETAILED DESCRIPTION OF THE EMBODIMENTS

The present disclosure provides a method for protein-protein docking based on identification of a low-entropy hydration layer on a protein surface, and belongs to a novel method for predicting docking sites of the protein. The method for protein-protein docking based on identification of a low-entropy hydration layer on a protein surface follows these guidelines:
1, if the amino acids of the protein surface are selected from the group consisting of leucine (Leu), tyrosine (Tyr), tryptophan (Trp), isoleucine (Ile), methionine (Met), phenylalanine (Phe), arginine (Arg), and lysine (Lys), and when carbonyl oxygen atoms or amide nitrogen atoms on backbone of the amino acids are exposed on the protein surface, changing the carbonyl oxygen atoms or the amide nitrogen atoms on the backbone to hydrophobic atoms, indicating that hydrophilic oxygen and nitrogen atoms of the backbone of residue Leu, Tyr, Trp, Ile, Met, Phe, Arg, or Lys become the hydrophobic atoms;
2, if the amino acids of the protein surface belong to residue Trp, Tyr, Lys, and Met, changing oxygen and nitrogen atoms of residue side chains of the amino acids to the hydrophobic carbon atoms, indicating that the hydrophilic oxygen and nitrogen atoms at a head of side chains of residue Trp, Tyr, Lys, and Met become the hydrophobic atoms; and
3, if hydrogen bonds are formed between the oxygen and nitrogen atoms of the protein surface, whether in an “α-helix” and a “β-sheet” of a secondary structure of the protein or in elsewhere, changing the oxygen and nitrogen atoms that form the hydrogen bonds to the hydrophobic atoms.

Without displaying hydrogen atoms, distribution of the low-entropy hydration layer region on the protein surface is obtained by the above principles, and an area and a shape of the low-entropy hydration layer region are determined by a computer program, so as to obtain a low-entropy hydration layer sample with a larger area. By comparing the area and shape matching of the low-entropy hydration layers of two protein samples, these samples are compared overlappingly, and two samples are obtained whose graphic contours of the low-entropy hydration layer are closest to the two protein samples using a search algorithm. By docking the two proteins according to obtained contour lines, it is verified that whether space contours of a docking surface of the two proteins match. If the space contours match, a docking position model of the proteins can be obtained.

The following describes the present disclosure in detail with reference to specific implementations.

### Specific Implementation 1

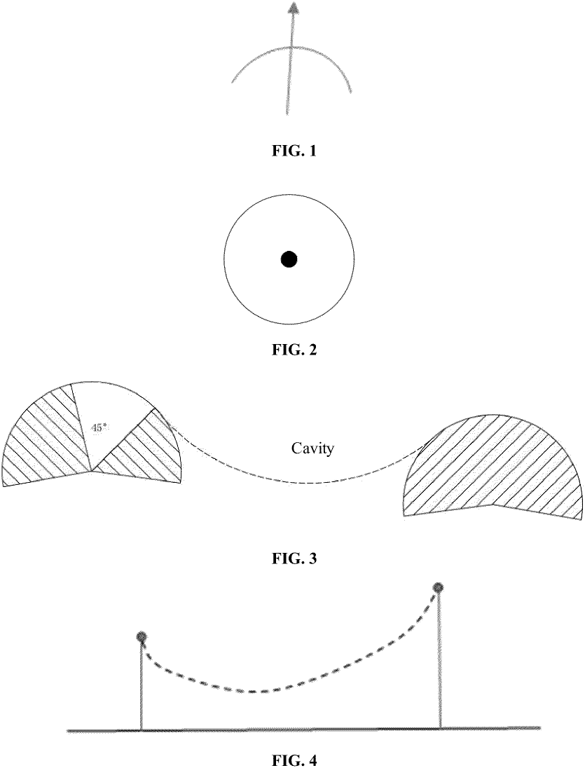

The present implementation provides a method for protein-protein docking based on identification of a low-entropy hydration layer on a protein surface, including the following steps:
step 1, rubbing heavy atoms on a protein surface, where the heavy atoms include carbon atoms, nitrogen atoms, oxygen atoms, and sulfur atoms; calculating an average spatial coordinate of the heavy atoms in the protein, and decomposing the protein into 20 pieces in a direction of a rubbing area using the average spatial coordinate as a center of projection;
reading data information in a protein data bank (PDB) structure file of the protein, to obtain a three-dimensional spatial coordinate of each heavy atom on the protein; and
dividing the protein atoms into the 20 regions with the 20 vertices of the regular dodecahedron as follows: the regular dodecahedron has the 20 vertices, and the average spatial coordinate of all atoms of the protein is used as a spatial origin, namely the center of projection; if in a spatial area, an angle (an included angle between two vertex vectors) is less than 41° between a vector pointing to each atom from the spatial origin and a vector pointing to the vertex, the atom is divided into this spatial area; the 20 vertices of the regular dodecahedron divide the protein atoms into 20 areas, and one divided area of the protein surface is regarded as one rubbing area;
step 2, for a protein in each rubbing area, conducting projection from the projection center to the rubbing area; taking a region of the projection center to vertices of the regular dodecahedron as a z-axis, conducting projection on z-coordinates of all the atoms of the protein in the region onto the z-axis; selecting externally lateral atoms on 30% of the maximum distance z from the center of projection to the protein atoms on the z-axis as the surface atoms of the protein, that is, selecting atoms with a distance from the center of projection of greater than 70% × d_{surface} as the surface atoms of the protein, where the d_{surface} is a distance from the center of projection to a maximum z-coordinate of the atoms of the protein surface on the z-axis; where for different proteins, a distance from the center of projection to the vertex is different, and the program can automatically calculate the distance according to actual situations;
step 3, traversing the surface atoms, and changing a hydrophobicity and a hydrophilicity of the atoms according to amino acids and the surface atoms of the protein surface:
   case 1: if the amino acids of the protein surface are selected from the group consisting of leucine (Leu), tyrosine (Tyr), tryptophan (Trp), isoleucine (Ile), methionine (Met), phenylalanine (Phe), arginine (Arg), and lysine (Lys), and when carbonyl oxygen atoms or amide nitrogen atoms on backbone of the amino acids are exposed on the protein surface, changing the carbonyl oxygen atoms or the amide nitrogen atoms on the backbone to hydrophobic atoms, indicating that hydrophilic oxygen and nitrogen atoms of the backbone of the Leu, the Tyr, the Trp, the Ile, the Met, the Phe, the Arg, or the Lys become the hydrophobic atoms;
   case 2: if the amino acids of the protein surface belong to the Trp, the Tyr, the Lys, and the Met, changing oxygen and nitrogen atoms of side chains of the amino acids to the hydrophobic atoms, indicating that the hydrophilic oxygen and nitrogen atoms at a head of the side chains of the Trp, the Tyr, the Lys, and the Met become the hydrophobic atoms; and
   case 3: if hydrogen bonds are formed between the oxygen and nitrogen atoms of the protein surface, whether in an “α-helix” and a “β-sheet” of a secondary structure of the protein or in elsewhere, changing the oxygen and nitrogen atoms that form the hydrogen bonds to the hydrophobic atoms;
step 4, after changing the hydrophobicity and the hydrophilicity of the atoms in the 20 regions of the protein in step 3, re-fitting a plane by a least square method to the surface atoms of the protein in each of the 20 regions, as a candidate protein docking plane; calculating an average distance di between a central coordinate position (xi, yi) of each of the protein docking planes in the 20 regions and all atoms on the plane, where i is a serial number of the rubbing area;
after fitting 20 planes, comparing obtained planes and excluding duplicate planes; during excluding the duplicate planes, if two fitting planes have an included angle of less than or equal to 10° from a spatial origin to vertical vectors of the respective planes, regarding the two fitting planes as a same plane; and
denoting remaining fitting planes as surface planes, and for each surface plane, selecting a central atom in a hydrophobic connection region, and marking atoms in the hydrophobic connection region; where a method includes the following steps:
transforming atomic coordinates into a three-dimensional space with the surface plane as an xy plane by coordinate transformation; taking xy coordinates of the atomic coordinates (equivalent to compression to the xy plane), if in a circle centered on oxygen and nitrogen atoms and with a radius of 5 angstroms, there are no other oxygen and nitrogen atoms, not using this atom as a boundary in a subsequent search for the boundary; otherwise, in the xy plane (two-dimensional plane), with oxygen and nitrogen atoms as the boundaries, assigning carbon and sulfur atoms as 1 in a circle centered on corresponding oxygen and nitrogen atoms and with a radius of 3 angstroms; taking assigned carbon and sulfur atoms as a center, if there are unassigned carbon and sulfur atoms in a circle centered on the assigned carbon and sulfur atoms and with a radius of 3 angstroms, adding up values of the assigned atoms in a circle centered on the unassigned carbon and sulfur atoms and with a radius of 3 angstroms as values of the unassigned carbon and sulfur atoms; at this time, only calculating the values of the unassigned carbon and sulfur atoms, and recording as pseudo-valuation of the unassigned carbon and sulfur atoms, while not assigning the unassigned carbon and sulfur atoms; when all atoms are searched for one round, assigning the pseudo-valuation of the unassigned carbon and sulfur atoms to the corresponding unassigned carbon and sulfur atoms, and starting a new round of assignment until all carbon and sulfur atoms complete the assignment; and
for each carbon and sulfur atom, in a circle centered on the carbon and sulfur atoms and with a radius of 3 angstroms, if values of the carbon and sulfur atoms are greater than or equal to values of surrounding atoms, regarding the carbon and sulfur atoms as central atoms of the corresponding hydrophobic connection region;
taking the central atom of the hydrophobic connection region as a center and 10° as a step size, dividing an area around the central atom, and selecting atoms with a value of greater than or equal to 3 in a fan-shaped area corresponding to each 10°; when an atom with a value of less than 3 appears for the first time (since the central atom is used as a center of the divided area, values of the atoms decrease progressively in a direction from the center to an opening of each fan-shaped area), selecting a distance from an atom with a value of 3 closest to the atom with a value of less than 3 to the center as a cut-off distance; and selecting atoms within the cut-off distance as atoms within the hydrophobic connection region;
step 5, calculating a hydrophobic area of the hydrophobic atoms on a surface of each hydrophobic connection region separately; a method includes the following steps:
   step 5.1, for the surface heavy atoms of the protein in each hydrophobic connection region, displaying the surface heavy atoms as a sphere with an action radius of 1.8 angstroms; with each surface heavy atom as a center, making a hemisphere in a projection direction, where the hemisphere is a hemispherical shell, as shown in FIG. 1 and FIG. 2;
   step 5.2, establishing a two-dimensional grid with an interval of 0.1 angstrom on a plane of the hydrophobic connection region, and recording height information and a heavy atom type of the corresponding protein surface in each two-dimensional grid; and
   step 5.3, establishing a surface with a radius of 1.8 angstroms as an action radius of the heavy atoms, with possibility of voids and discontinuities on the surface, where the hemispherical shells of the two heavy atoms have no intersection, and a distance between the two atoms is less than 6 angstroms, as shown in FIG. 3; taking a connection between a fan-shaped region formed by the hemispherical shell of one heavy atom with a projection direction in an included angle of 45° and a fan-shaped region formed by the hemispherical shell of another heavy atom with the projection direction in an included angle of 45° as an interpolation area; taking intersections of the connection and the two fan-shaped regions formed by the hemispherical shells of the two heavy atoms with the projection direction in an included angle of 45° as interpolation endpoints, obtaining a plane of a cavity between the hemispherical shells of the two heavy atoms by an interpolation, and abbreviating the plane of the cavity as an interpolation plane, as shown in FIG. 4; and
at the cavity, selecting a surface of the heavy atom at 45° to the projection direction as a surface connection point, and conducting cubic spline interpolation to obtain a type of the heavy atom and a three-dimensional height of the interpolation at the cavity, and then determining a space area corresponding to each grid in the two-dimensional grid, and then determining an area of the hydrophobic connection region (space area); where
if the heavy atoms on both sides of the interpolation plane have a same type, the hydrophobic/hydrophilic types of the interpolation plane are the same as those on both sides; and if the heavy atoms on both sides of the interpolation plane have different types, using the middle as a boundary, the hydrophobic/hydrophilic types are different on both sides;
step 6, selecting first three hydrophobic connection regions with a maximum hydrophobic area in the protein as possible docking positions, and docking two proteins to be docked, as follows:
   denoting the hydrophobic connection regions of the two proteins to be docked as a hydrophobic connection region A and a hydrophobic connection region B, respectively; determining a search range on the hydrophobic connection region A or the hydrophobic connection region B according to a relatively high average distance d=max(di, dj) corresponding to the two hydrophobic connection regions;
   fixing the hydrophobic connection region A, establishing a 2d × 2d two-dimensional grid with an interval of 3 angstroms using a center coordinate of the hydrophobic connection region A as a surface origin of the hydrophobic connection region, and denoting area boundaries of the search range corresponding to the 2d × 2d of the hydrophobic connection region A as (x_max, y_max); locating a center coordinate of the hydrophobic connection region B in grid points of the two-dimensional grid in sequence, and rotating at an interval of 5° and calculating a docking situation of the two hydrophobic connection regions at each grid point; a calculation method includes the following steps:
      taking a normal vector of a fitting plane of each of the hydrophobic connection region A and the hydrophobic connection region B as a z-axis of the fitting plane, making the two hydrophobic connection regions approach on the z-axis of the fitting plane, placing the hydrophobic connection region B above the hydrophobic connection region A, and gradually reducing a height of the hydrophobic connection region B, that is, making the hydrophobic connection region B gradually approach the hydrophobic connection region A; where making the two hydrophobic connection regions gradually approach on the plane is to gradually overlap the z-axes of the two hydrophobic connection regions; however, if the z-axis of the hydrophobic connection region A faces upwards, the z-axis of the hydrophobic connection region B faces downwards, which is similar to conducting a coordinate transformation on the hydrophobic connection region B; and after the transformation, the two areas are in a same coordinate system;
      according to the process of the hydrophobic connection region B gradually approaching the hydrophobic connection region A, setting a minimum distance to be 1 angstrom between the atoms on the hydrophobic connection region A and the hydrophobic connection region B, and denoting a position of the minimum distance as a space docking position; in the space docking position, finding out minimum x- and y-axis coordinates of the atomic coordinates of the hydrophobic connection region A and the hydrophobic connection region B, denoting as (x_min, y_min), so as to facilitate the subsequent grid establishment and docking calculation;
      after the coordinate (x_min, y_min) are obtained, pressing the atoms in the hydrophobic connection regions A and B to an x-y plane, calculating atoms closest to the coordinate (x_min, y_min) in the hydrophobic connection regions A and B in the x-y plane, respectively, denoting two obtained atoms as an atom a and an atom b, taking an average of z-axis heights corresponding to respective spatial coordinates represented by the atom a and the atom b under the space docking position as a docking plane z value, and then determining a three-dimensional spatial coordinate (x_min, y_min, z); calculating a real distance from the atom a and the atom b in the space docking position separately using the coordinate (x_min, y_min, z), and if there is a distance of not less than 6 angstroms in the two distances, determining that the hydrophobic connection region A and the hydrophobic connection region B have no docking surface at the coordinate (only not at the current coordinate, but there is definitely a docking surface existing during the search); otherwise, determining that there is a docking surface between two proteins at the coordinate;
      if there is no docking surface at the current space docking position, moving the hydrophobic connection region B on the two-dimensional grid of the hydrophobic connection region A, with a movement step size of (x, y) by 0.1 angstrom, that is: y increases by 0.1 once, and x goes from x_min to x_max; and y increases by 0.1 once more, and x goes from x_min to x_max once more; repeating the above step, with a range of y changing from y_min to y_max, until all coordinates are traversed;
      when there is a docking surface between the two proteins, recording a type of the docking surface under the space docking position (carbon atom-carbon atom, carbon atom-oxygen and nitrogen atoms, and oxygen and nitrogen atoms-oxygen and nitrogen atoms);
      when there is a docking surface between the two proteins, determining a docking type at a docking interface of the hydrophobic connection regions A and B under the current space docking position, and adjusting the docking plane z value to a z-axis height corresponding to a same distance from the atom a and the atom b; where
      the docking type includes carbon atom-carbon atom, carbon atom-oxygen and nitrogen atoms, and oxygen and nitrogen atoms-oxygen and nitrogen atoms, and areas of different docking types at the docking interface of the hydrophobic connection regions A and B are calculated according to the docking type;
      if there are multiple docking positions, a maximum complete docking area is selected as the docking position, and a complete docking area of the two proteins is integrally calculated at the position.

### Example

In the present disclosure, based on related researches, a low-entropy hydration layer matching mechanism was proposed to identify protein docking sites. This mechanism revealed the mechanism of protein docking based on hydrophobic interaction. It was found by experiments that the hydrophobic interaction binding force between a COVID-19 virus S protein and an angiotensin converting enzyme ACE2 protein was much greater than that of a homologous SARS virus S protein and the ACE2 protein. The low-entropy hydration layer matching mechanism was consistent with results of an experiment published in “Science” and reasonably explained the experiment. This study showed that a reason why the COVID-19 virus was super infectious was that a maximum low-entropy hydration layer region on a surface of its S protein and a maximum low-entropy hydration layer region on a surface of its receptor protein had a high degree of matching, causing the COVID-19 virus to express an extraordinary receptor binding capacity, namely a super infectivity. Relevant studies had shown that the protein docking, including the COVID-19 virus, was dominated by hydrophobic forces, and hydrogen bonding and biochemical reactions between proteins were facilitated by mutual attraction of the maximum low-entropy hydration layers.

For protein-protein structure docking, an optimal binding site could be obtained by the distribution of low-entropy hydration layer regions on a surface of the protein structure. By analyzing three-dimensional images of the low-entropy hydration layer regions of the docking positions between hundreds of proteins, the images were compared with several maximum low-entropy hydration layer regions on the entire protein surface identified by a computer program. It was found that among several three-dimensional areas of the low-entropy hydration layers identified by the computer program, a projected area perfectly matching the low-entropy hydration layer could be found at an actual docking position. That is to say, hydrophobic docking sites between subunit structures were successfully predicted, which were fully in line with a theory that the maximum low-entropy hydration layer matching mechanism dominated the docking between protein structures to form protein-protein interactions. The results of program verification showed that the low-entropy hydration layer matching mechanism could accurately predict the docking position between protein-protein structures (referring to accompanying drawings in the description), demonstrating that the low-entropy hydration layer matching mechanism dominated by hydrophobic interactions drove the formation of protein-protein docking structures.

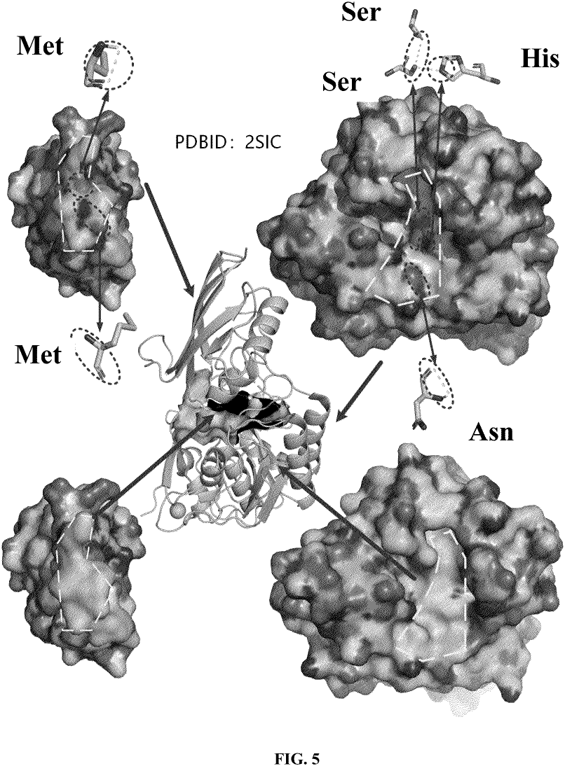

The method for protein-protein docking based on identification of a low-entropy hydration layer on a protein surface was described using the protein-protein docking of PDBID:2SIC as an example. The docking effect was shown in FIG. 5, where a middle figure represented the protein-protein docking, and a cyan part was a docking surface of the low-entropy hydration layer; upper two figures represented amino acids after PDBID:2SIC being manually manipulated to discolor according to discoloration criteria identified by the protein low-entropy hydration layer; and lower two figures showed that after treating by the discoloration criteria identified by the protein low-entropy hydration layer, the cyan part was a surface of the low-entropy hydration layer where the two proteins are docked.

### Specific Implementation 2

The present implementation provides a device for protein-protein docking based on identification of a low-entropy hydration layer on a protein surface, including a memory, further including a processor, where the memory stores at least one instruction, and the at least one instruction is loaded and executed by the processor to implement the method for protein-protein docking based on identification of a low-entropy hydration layer on a protein surface.

When the device includes only the memory, the device may be the memory itself.

The present disclosure may also have many other embodiments. Without departing from the spirit and essence of the present disclosure, those skilled in the art can make various corresponding changes and modifications according to the present disclosure, and these corresponding changes and modifications should belong to the protection scope of the appended claims of the present disclosure.

What is claimed is:

1. A method for protein-protein docking based on identification of a low-entropy hydration layer on a protein surface, comprising the following steps:
step 1, rubbing heavy atoms on a protein surface, wherein the heavy atoms comprise carbon atoms, nitrogen atoms, oxygen atoms, and sulfur atoms; calculating an average spatial coordinate of the heavy atoms in the protein, and dividing protein atoms into 20 regions with 20 vertices of a regular dodecahedron and using the average spatial coordinate as a center of projection;
step 2, for the protein in each obtained rubbing area, using a direction of the center of projection to the vertices of the regular dodecahedron as a z-axis, and determining surface atoms of the protein according to a maximum distance z from the center of projection to the protein atoms on the z-axis;
step 3, traversing the surface atoms, and changing a hydrophobicity and a hydrophilicity of the atoms according to amino acids and the surface atoms of the protein surface, wherein
case 1: if the amino acids of the protein surface are selected from the group consisting of leucine (Leu), tyrosine (Tyr), tryptophan (Trp), isoleucine (Ile), methionine (Met), phenylalanine (Phe), arginine (Arg), and lysine (Lys), and when carbonyl oxygen atoms or amide nitrogen atoms on backbone of the amino acids are exposed on the protein surface, changing the carbonyl oxygen atoms or the amide nitrogen atoms on the backbone to hydrophobic atoms, indicating that hydrophilic oxygen and nitrogen atoms of the backbone of residue Leu, Tyr, Trp, Ile, Met, Phe, Arg, or Lys become the hydrophobic atoms;
case 2: if the amino acids of the protein surface belong to residue Trp, Tyr, Lys, and Met, changing oxygen and nitrogen atoms of residue side chains of the amino acids to the hydrophobic atoms, indicating that the hydrophilic oxygen and nitrogen atoms at a head of the side chains of residue Trp, Tyr, Lys, and Met become the hydrophobic atoms; and
case 3: if hydrogen bonds are formed between the oxygen and nitrogen atoms of the protein surface, whether in an “a-helix” and a “β-sheet” of a secondary structure of the protein or in elsewhere, changing the oxygen and nitrogen atoms that form the hydrogen bonds to the hydrophobic atoms;
step 4, after changing the hydrophobicity and the hydrophilicity of the atoms in the 20 regions of the protein in step 3, re-fitting a plane by a least square method to the surface atoms of the protein in each of the 20 regions, as a candidate protein docking plane; calculating an average distance di between a central coordinate position (xi, yi) of each of the protein docking planes in the 20 regions and all atoms on the plane, wherein i is a serial number of the rubbing area;
after fitting 20 planes, comparing obtained planes and excluding duplicate planes; during excluding the duplicate planes, if two fitting planes have an included angle of less than or equal to 10° from a spatial origin to vertical vectors of the respective planes, regarding the two fitting planes as a same plane; and
denoting remaining fitting planes as surface planes, and for each surface plane, selecting a central atom in a hydrophobic connection region, and marking atoms in the hydrophobic connection region;
step 5, calculating a hydrophobic area of the hydrophobic atoms on a surface of each hydrophobic connection region separately; and
step 6, selecting first three hydrophobic connection regions with a maximum hydrophobic area in the protein as possible docking positions, and docking two proteins to be docked.

2. The method for protein-protein docking based on identification of a low-entropy hydration layer on a protein surface according to claim 1, wherein step 1 specifically comprises:
reading data information in a protein data bank (PDB) structure file of the protein, to obtain a three-dimensional spatial coordinate of each heavy atom on the protein surface; and
dividing the protein atoms into the 20 regions with the 20 vertices of the regular dodecahedron as follows: the regular dodecahedron has the 20 vertices, and the average spatial coordinate of all atoms of the protein is used as a spatial origin; if in a spatial area, an angle is less than 41° between a vector pointing to each atom from the spatial origin and a vector pointing to the vertex, the atom is divided into this spatial area; the 20 vertices of the regular dodecahedron divide the protein atoms into 20 areas, and one divided area of the protein surface is regarded as one rubbing area.

3. The method for protein-protein docking based on identification of a low-entropy hydration layer on a protein surface according to claim 2, wherein in step 2, a process of determining the surface atoms of the protein according to the maximum distance z from the center of projection to the protein atoms on the z-axis specifically comprises:
selecting externally lateral atoms on 30% of the maximum distance z from the center of projection to the protein atoms on the z-axis as the surface atoms of the protein, that is, selecting atoms with a distance from the center of projection of greater than 70% × d_{surface} as the surface atoms of the protein, wherein the d_{surface} is a distance from the center of projection to a maximum z-coordinate of the atoms of the protein surface on the z-axis.

4. The method for protein-protein docking based on identification of a low-entropy hydration layer on a protein surface according to claim 1, wherein in step 4, a process of denoting the remaining fitting planes as the surface planes, and for each surface plane, selecting the central atom in the hydrophobic connection region specifically comprises:
transforming atomic coordinates into a three-dimensional space with the surface plane as an xy plane by coordinate transformation; taking xy coordinates of the atomic coordinates, if in a circle centered on oxygen and nitrogen atoms and with a radius of 5 angstroms, there are no other oxygen and nitrogen atoms, not using this atom as a boundary in a subsequent search for the boundary; otherwise, in the xy plane, with oxygen and nitrogen atoms as the boundaries, assigning carbon and sulfur atoms as 1 in a circle centered on corresponding oxygen and nitrogen atoms and with a radius of 3 angstroms; taking assigned carbon and sulfur atoms as a center, if there are unassigned carbon and sulfur atoms in a circle centered on the assigned carbon and sulfur atoms and with a radius of 3 angstroms, adding up values of the assigned atoms in a circle centered on the unassigned carbon and sulfur atoms and with a radius of 3 angstroms as values of the unassigned carbon and sulfur atoms; at this time, only calculating the values of the unassigned carbon and sulfur atoms, and recording as pseudo-valuation of the unassigned carbon and sulfur atoms, while not assigning the unassigned carbon and sulfur atoms; when all atoms are searched for one round, assigning the pseudo-valuation of the unassigned carbon and sulfur atoms to the corresponding unassigned carbon and sulfur atoms, and starting a new round of assignment until all carbon and sulfur atoms complete the assignment; and
for each carbon and sulfur atom, in a circle centered on the carbon and sulfur atoms and with a radius of 3 angstroms, if values of the carbon and sulfur atoms are greater than or equal to values of surrounding atoms, regarding the carbon and sulfur atoms as central atoms of the corresponding hydrophobic connection region.

5. The method for protein-protein docking based on identification of a low-entropy hydration layer on a protein surface according to claim 2, wherein in step 4, a process of denoting the remaining fitting planes as the surface planes, and for each surface plane, selecting the central atom in the hydrophobic connection region specifically comprises:
transforming atomic coordinates into a three-dimensional space with the surface plane as an xy plane by coordinate transformation; taking xy coordinates of the atomic coordinates, if in a circle centered on oxygen and nitrogen atoms and with a radius of 5 angstroms, there are no other oxygen and nitrogen atoms, not using this atom as a boundary in a subsequent search for the boundary; otherwise, in the xy plane, with oxygen and nitrogen atoms as the boundaries, assigning carbon and sulfur atoms as 1 in a circle centered on corresponding oxygen and nitrogen atoms and with a radius of 3 angstroms; taking assigned carbon and sulfur atoms as a center, if there are unassigned carbon and sulfur atoms in a circle centered on the assigned carbon and sulfur atoms and with a radius of 3 angstroms, adding up values of the assigned atoms in a circle centered on the unassigned carbon and sulfur atoms and with a radius of 3 angstroms as values of the unassigned carbon and sulfur atoms; at this time, only calculating the values of the unassigned carbon and sulfur atoms, and recording as pseudo-valuation of the unassigned carbon and sulfur atoms, while not assigning the unassigned carbon and sulfur atoms; when all atoms are searched for one round, assigning the pseudo-valuation of the unassigned carbon and sulfur atoms to the corresponding unassigned carbon and sulfur atoms, and starting a new round of assignment until all carbon and sulfur atoms complete the assignment; and
for each carbon and sulfur atom, in a circle centered on the carbon and sulfur atoms and with a radius of 3 angstroms, if values of the carbon and sulfur atoms are greater than or equal to values of surrounding atoms, regarding the carbon and sulfur atoms as central atoms of the corresponding hydrophobic connection region.

6. The method for protein-protein docking based on identification of a low-entropy hydration layer on a protein surface according to claim 3, wherein in step 4, a process of denoting the remaining fitting planes as the surface planes, and for each surface plane, selecting the central atom in the hydrophobic connection region specifically comprises:
transforming atomic coordinates into a three-dimensional space with the surface plane as an xy plane by coordinate transformation; taking xy coordinates of the atomic coordinates, if in a circle centered on oxygen and nitrogen atoms and with a radius of 5 angstroms, there are no other oxygen and nitrogen atoms, not using this atom as a boundary in a subsequent search for the boundary; otherwise, in the xy plane, with oxygen and nitrogen atoms as the boundaries, assigning carbon and sulfur atoms as 1 in a circle centered on corresponding oxygen and nitrogen atoms and with a radius of 3 angstroms; taking assigned carbon and sulfur atoms as a center, if there are unassigned carbon and sulfur atoms in a circle centered on the assigned carbon and sulfur atoms and with a radius of 3 angstroms, adding up values of the assigned atoms in a circle centered on the unassigned carbon and sulfur atoms and with a radius of 3 angstroms as values of the unassigned carbon and sulfur atoms; at this time, only calculating the values of the unassigned carbon and sulfur atoms, and recording as pseudo-valuation of the unassigned carbon and sulfur atoms, while not assigning the unassigned carbon and sulfur atoms; when all atoms are searched for one round, assigning the pseudo-valuation of the unassigned carbon and sulfur atoms to the corresponding unassigned carbon and sulfur atoms, and starting a new round of assignment until all carbon and sulfur atoms complete the assignment; and
for each carbon and sulfur atom, in a circle centered on the carbon and sulfur atoms and with a radius of 3 angstroms, if values of the carbon and sulfur atoms are greater than or equal to values of surrounding atoms, regarding the carbon and sulfur atoms as central atoms of the corresponding hydrophobic connection region.

7. The method for protein-protein docking based on identification of a low-entropy hydration layer on a protein surface according to claim 4, wherein in step 4, a process of marking atoms in the hydrophobic connection region specifically comprises:
taking the central atom of the hydrophobic connection region as a center and 10° as a step size, dividing an area around the central atom, and selecting atoms with a value of greater than or equal to 3 in a fan-shaped area corresponding to each 10°; when an atom with a value of less than 3 appears for the first time, selecting a distance from an atom with a value of 3 closest to the atom with a value of less than 3 to the center as a cut-off distance; and selecting atoms within the cut-off distance as atoms within the hydrophobic connection region.

8. The method for protein-protein docking based on identification of a low-entropy hydration layer on a protein surface according to claim 7, wherein in step 5, a process of calculating the hydrophobic area of the hydrophobic atoms on the surface of each hydrophobic connection region separately specifically comprises:
step 5.1, for the surface heavy atoms of the protein in each hydrophobic connection region, displaying the surface heavy atoms as a sphere with an action radius of 1.8 angstroms; with each surface heavy atom as a center, making a hemisphere in a projection direction, wherein the hemisphere is a hemispherical shell;
step 5.2, establishing a two-dimensional grid with an interval of 0.1 angstrom on a plane of the hydrophobic connection region, and recording height information and a heavy atom type of the corresponding protein surface in each two-dimensional grid; and
step 5.3, establishing a surface with a radius of 1.8 angstroms as an action radius of the heavy atoms, with possibility of voids and discontinuities on the surface, wherein the hemispherical shells of the two heavy atoms have no intersection, and a distance between the two atoms is less than 6 angstroms, as shown in FIG. 3; taking a connection between a fan-shaped region formed by the hemispherical shell of one heavy atom with a projection direction in an included angle of 45° and a fan-shaped region formed by the hemispherical shell of another heavy atom with the projection direction in an included angle of 45° as an interpolation area; taking intersections of the connection and the two fan-shaped regions formed by the hemispherical shells of the two heavy atoms with the projection direction in an included angle of 45° as interpolation endpoints, obtaining a plane of a cavity between the hemispherical shells of the two heavy atoms by an interpolation, and abbreviating the plane of the cavity as an interpolation plane, as shown in FIG. 4; and
at the cavity, selecting a surface of the heavy atom at 45° to the projection direction as a surface connection point, and conducting cubic spline interpolation to obtain a type of the heavy atom and a three-dimensional height of the interpolation at the cavity, and then determining a space area corresponding to each grid in the two-dimensional grid, and then determining an area of the hydrophobic connection region.

9. The method for protein-protein docking based on identification of a low-entropy hydration layer on a protein surface according to claim 8, wherein in step 6, a process of selecting the first three hydrophobic connection regions with a maximum hydrophobic area in the protein as the possible docking positions, and docking the two proteins to be docked specifically comprises:
denoting the hydrophobic connection regions of the two proteins to be docked as a hydrophobic connection region A and a hydrophobic connection region B, respectively; determining a search range on the hydrophobic connection region A or the hydrophobic connection region B according to a relatively high average distance d=max(di, dj) corresponding to the two hydrophobic connection regions;
fixing the hydrophobic connection region A, establishing a 2d × 2d two-dimensional grid with an interval of 3 angstroms using a center coordinate of the hydrophobic connection region A as a surface origin of the hydrophobic connection region, and denoting area boundaries of the search range corresponding to the 2d × 2d of the hydrophobic connection region A as (x_max, y_max); locating a center coordinate of the hydrophobic connection region B in grid points of the two-dimensional grid in sequence, and rotating at an interval of 5° and calculating a docking situation of the two hydrophobic connection regions at each grid point; a calculation method comprises the following steps:
taking a normal vector of a fitting plane of each of the hydrophobic connection region A and the hydrophobic connection region B as a z-axis of the fitting plane, making the two hydrophobic connection regions approach on the z-axis of the fitting plane, placing the hydrophobic connection region B above the hydrophobic connection region A, and gradually reducing a height of the hydrophobic connection region B, that is, making the hydrophobic connection region B gradually approach the hydrophobic connection region A; wherein making the two hydrophobic connection regions gradually approach on the plane is to gradually overlap the z-axes of the two hydrophobic connection regions; however, if the z-axis of the hydrophobic connection region A faces upwards, the z-axis of the hydrophobic connection region B faces downwards, which is similar to conducting a coordinate transformation on the hydrophobic connection region B; and after the transformation, the two areas are in a same coordinate system;
according to the process of the hydrophobic connection region B gradually approaching the hydrophobic connection region A, setting a minimum distance to be 1 angstrom between the atoms on the hydrophobic connection region A and the hydrophobic connection region B, and denoting a position of the minimum distance as a space docking position; in the space docking position, finding out minimum x- and y-axis coordinates of the atomic coordinates of the hydrophobic connection region A and the hydrophobic connection region B, denoting as (x_min, y_min), so as to facilitate the subsequent grid establishment and docking calculation;
after the coordinate (x_min,y_min) are obtained, pressing the atoms in the hydrophobic connection regions A and B to an x-y plane, calculating atoms closest to the coordinate (x_min, y_min) in the hydrophobic connection regions A and B in the x-y plane, respectively, denoting two obtained atoms as an atom a and an atom b, taking an average of z-axis heights corresponding to respective spatial coordinates represented by the atom a and the atom b under the space docking position as a docking plane z value, and then determining a three-dimensional spatial coordinate (x_min,y_min, z); calculating a real distance from the atom a and the atom b in the space docking position separately using the coordinate (x_min,y_min, z), and if there is a distance of not less than 6 angstroms in the two distances, determining that the hydrophobic connection region A and the hydrophobic connection region B have no docking surface at the coordinate; otherwise, determining that there is a docking surface between two proteins at the coordinate;
if there is no docking surface at the current space docking position, moving the hydrophobic connection region B on the two-dimensional grid of the hydrophobic connection region A, with a movement step size of (x, y) by 0.1 angstrom, that is: y increases by 0.1 once, and x goes from x_min to x_max; and y increases by 0.1 once more, and x goes from x_min to x_max once more; repeating the above step, with a range of y changing from y_min to y_max, until all coordinates are traversed;
when there is a docking surface between the two proteins, recording a type of the docking surface under the space docking position;
when there is a docking surface between the two proteins, determining a docking type at a docking interface of the hydrophobic connection regions A and B under the current space docking position, and adjusting the docking plane z value to a z-axis height corresponding to a same distance from the atom a and the atom b; wherein
the docking type comprises carbon atom-carbon atom, carbon atom-oxygen and nitrogen atoms, and oxygen and nitrogen atoms-oxygen and nitrogen atoms, and areas of different docking types at the docking interface of the hydrophobic connection regions A and B are calculated according to the docking type.

10. The method for protein-protein docking based on identification of a low-entropy hydration layer on a protein surface according to claim 9, wherein in step 6, if there are multiple docking positions, a maximum complete docking area is selected as the docking position, and a complete docking area of the two proteins is integrally calculated at the position.

11. A device for protein-protein docking based on identification of a low-entropy hydration layer on a protein surface, comprising a memory, wherein the memory stores at least one instruction, and the at least one instruction is loaded and executed by a processor to implement the method for protein-protein docking based on identification of a low-entropy hydration layer on a protein surface according to claim 1.

12. The device for protein-protein docking based on identification of a low-entropy hydration layer on a protein surface according to claim 11, wherein step 1 specifically comprises:
reading data information in a protein data bank (PDB) structure file of the protein, to obtain a three-dimensional spatial coordinate of each heavy atom on the protein surface; and
dividing the protein atoms into the 20 regions with the 20 vertices of the regular dodecahedron as follows: the regular dodecahedron has the 20 vertices, and the average spatial coordinate of all atoms of the protein is used as a spatial origin; if in a spatial area, an angle is less than 41° between a vector pointing to each atom from the spatial origin and a vector pointing to the vertex, the atom is divided into this spatial area; the 20 vertices of the regular dodecahedron divide the protein atoms into 20 areas, and one divided area of the protein surface is regarded as one rubbing area.

13. The device for protein-protein docking based on identification of a low-entropy hydration layer on a protein surface according to claim 12, wherein in step 2, a process of determining the surface atoms of the protein according to the maximum distance z from the center of projection to the protein atoms on the z-axis specifically comprises:
selecting externally lateral atoms on 30% of the maximum distance z from the center of projection to the protein atoms on the z-axis as the surface atoms of the protein, that is, selecting atoms with a distance from the center of projection of greater than 70% × d_{surface} as the surface atoms of the protein, wherein the d_{surface} is a distance from the center of projection to a maximum z-coordinate of the atoms of the protein surface on the z-axis.

14. The device for protein-protein docking based on identification of a low-entropy hydration layer on a protein surface according to claim 11, wherein in step 4, a process of denoting the remaining fitting planes as the surface planes, and for each surface plane, selecting the central atom in the hydrophobic connection region specifically comprises:
transforming atomic coordinates into a three-dimensional space with the surface plane as an xy plane by coordinate transformation; taking xy coordinates of the atomic coordinates, if in a circle centered on oxygen and nitrogen atoms and with a radius of 5 angstroms, there are no other oxygen and nitrogen atoms, not using this atom as a boundary in a subsequent search for the boundary; otherwise, in the xy plane, with oxygen and nitrogen atoms as the boundaries, assigning carbon and sulfur atoms as 1 in a circle centered on corresponding oxygen and nitrogen atoms and with a radius of 3 angstroms; taking assigned carbon and sulfur atoms as a center, if there are unassigned carbon and sulfur atoms in a circle centered on the assigned carbon and sulfur atoms and with a radius of 3 angstroms, adding up values of the assigned atoms in a circle centered on the unassigned carbon and sulfur atoms and with a radius of 3 angstroms as values of the unassigned carbon and sulfur atoms; at this time, only calculating the values of the unassigned carbon and sulfur atoms, and recording as pseudo-valuation of the unassigned carbon and sulfur atoms, while not assigning the unassigned carbon and sulfur atoms; when all atoms are searched for one round, assigning the pseudo-valuation of the unassigned carbon and sulfur atoms to the corresponding unassigned carbon and sulfur atoms, and starting a new round of assignment until all carbon and sulfur atoms complete the assignment; and
for each carbon and sulfur atom, in a circle centered on the carbon and sulfur atoms and with a radius of 3 angstroms, if values of the carbon and sulfur atoms are greater than or equal to values of surrounding atoms, regarding the carbon and sulfur atoms as central atoms of the corresponding hydrophobic connection region.

15. The device for protein-protein docking based on identification of a low-entropy hydration layer on a protein surface according to claim 14, wherein in step 4, a process of marking atoms in the hydrophobic connection region specifically comprises:
taking the central atom of the hydrophobic connection region as a center and 10° as a step size, dividing an area around the central atom, and selecting atoms with a value of greater than or equal to 3 in a fan-shaped area corresponding to each 10°; when an atom with a value of less than 3 appears for the first time, selecting a distance from an atom with a value of 3 closest to the atom with a value of less than 3 to the center as a cut-off distance; and selecting atoms within the cut-off distance as atoms within the hydrophobic connection region.

16. The device for protein-protein docking based on identification of a low-entropy hydration layer on a protein surface according to claim 15, wherein in step 5, a process of calculating the hydrophobic area of the hydrophobic atoms on the surface of each hydrophobic connection region separately specifically comprises:
step 5.1, for the surface heavy atoms of the protein in each hydrophobic connection region, displaying the surface heavy atoms as a sphere with an action radius of 1.8 angstroms; with each surface heavy atom as a center, making a hemisphere in a projection direction, wherein the hemisphere is a hemispherical shell;
step 5.2, establishing a two-dimensional grid with an interval of 0.1 angstrom on a plane of the hydrophobic connection region, and recording height information and a heavy atom type of the corresponding protein surface in each two-dimensional grid; and
step 5.3, establishing a surface with a radius of 1.8 angstroms as an action radius of the heavy atoms, with possibility of voids and discontinuities on the surface, wherein the hemispherical shells of the two heavy atoms have no intersection, and a distance between the two atoms is less than 6 angstroms, as shown in FIG. 3; taking a connection between a fan-shaped region formed by the hemispherical shell of one heavy atom with a projection direction in an included angle of 45° and a fan-shaped region formed by the hemispherical shell of another heavy atom with the projection direction in an included angle of 45° as an interpolation area; taking intersections of the connection and the two fan-shaped regions formed by the hemispherical shells of the two heavy atoms with the projection direction in an included angle of 45° as interpolation endpoints, obtaining a plane of a cavity between the hemispherical shells of the two heavy atoms by an interpolation, and abbreviating the plane of the cavity as an interpolation plane, as shown in FIG. 4; and
at the cavity, selecting a surface of the heavy atom at 45° to the projection direction as a surface connection point, and conducting cubic spline interpolation to obtain a type of the heavy atom and a three-dimensional height of the interpolation at the cavity, and then determining a space area corresponding to each grid in the two-dimensional grid, and then determining an area of the hydrophobic connection region.

17. The device for protein-protein docking based on identification of a low-entropy hydration layer on a protein surface according to claim 16, wherein in step 6, a process of selecting the first three hydrophobic connection regions with a maximum hydrophobic area in the protein as the possible docking positions, and docking the two proteins to be docked specifically comprises:
denoting the hydrophobic connection regions of the two proteins to be docked as a hydrophobic connection region A and a hydrophobic connection region B, respectively; determining a search range on the hydrophobic connection region A or the hydrophobic connection region B according to a relatively high average distance d=max(di, dj) corresponding to the two hydrophobic connection regions;
fixing the hydrophobic connection region A, establishing a 2d × 2d two-dimensional grid with an interval of 3 angstroms using a center coordinate of the hydrophobic connection region A as a surface origin of the hydrophobic connection region, and denoting area boundaries of the search range corresponding to the 2d × 2d of the hydrophobic connection region A as (x_max, y_max); locating a center coordinate of the hydrophobic connection region B in grid points of the two-dimensional grid in sequence, and rotating at an interval of 5° and calculating a docking situation of the two hydrophobic connection regions at each grid point; a calculation method comprises the following steps:
taking a normal vector of a fitting plane of each of the hydrophobic connection region A and the hydrophobic connection region B as a z-axis of the fitting plane, making the two hydrophobic connection regions approach on the z-axis of the fitting plane, placing the hydrophobic connection region B above the hydrophobic connection region A, and gradually reducing a height of the hydrophobic connection region B, that is, making the hydrophobic connection region B gradually approach the hydrophobic connection region A; wherein making the two hydrophobic connection regions gradually approach on the plane is to gradually overlap the z-axes of the two hydrophobic connection regions; however, if the z-axis of the hydrophobic connection region A faces upwards, the z-axis of the hydrophobic connection region B faces downwards, which is similar to conducting a coordinate transformation on the hydrophobic connection region B; and after the transformation, the two areas are in a same coordinate system;
according to the process of the hydrophobic connection region B gradually approaching the hydrophobic connection region A, setting a minimum distance to be 1 angstrom between the atoms on the hydrophobic connection region A and the hydrophobic connection region B, and denoting a position of the minimum distance as a space docking position; in the space docking position, finding out minimum x- and y-axis coordinates of the atomic coordinates of the hydrophobic connection region A and the hydrophobic connection region B, denoting as (x_min, y_min), so as to facilitate the subsequent grid establishment and docking calculation;
after the coordinate (x_min,y_min) are obtained, pressing the atoms in the hydrophobic connection regions A and B to an x-y plane, calculating atoms closest to the coordinate (x_min, y_min) in the hydrophobic connection regions A and B in the x-y plane, respectively, denoting two obtained atoms as an atom a and an atom b, taking an average of z-axis heights corresponding to respective spatial coordinates represented by the atom a and the atom b under the space docking position as a docking plane z value, and then determining a three-dimensional spatial coordinate (x_min,y_min, z); calculating a real distance from the atom a and the atom b in the space docking position separately using the coordinate (x_min,y_min, z), and if there is a distance of not less than 6 angstroms in the two distances, determining that the hydrophobic connection region A and the hydrophobic connection region B have no docking surface at the coordinate; otherwise, determining that there is a docking surface between two proteins at the coordinate;
if there is no docking surface at the current space docking position, moving the hydrophobic connection region B on the two-dimensional grid of the hydrophobic connection region A, with a movement step size of (x, y) by 0.1 angstrom, that is: y increases by 0.1 once, and x goes from x_min to x_max; and y increases by 0.1 once more, and x goes from x_min to x_max once more; repeating the above step, with a range of y changing from y_min to y_max, until all coordinates are traversed;
when there is a docking surface between the two proteins, recording a type of the docking surface under the space docking position;
when there is a docking surface between the two proteins, determining a docking type at a docking interface of the hydrophobic connection regions A and B under the current space docking position, and adjusting the docking plane z value to a z-axis height corresponding to a same distance from the atom a and the atom b; wherein
the docking type comprises carbon atom-carbon atom, carbon atom-oxygen and nitrogen atoms, and oxygen and nitrogen atoms-oxygen and nitrogen atoms, and areas of different docking types at the docking interface of the hydrophobic connection regions A and B are calculated according to the docking type.

18. The device for protein-protein docking based on identification of a low-entropy hydration layer on a protein surface according to claim 17, wherein in step 6, if there are multiple docking positions, a maximum complete docking area is selected as the docking position, and a complete docking area of the two proteins is integrally calculated at the position.

19. The device for protein-protein docking based on identification of a low-entropy hydration layer on a protein surface according to claim 11, further comprising a processor, wherein the processor loads and executes the at least one instruction stored in the memory to implement the method for protein-protein docking based on identification of a low-entropy hydration layer on a protein surface.

20. The device for protein-protein docking based on identification of a low-entropy hydration layer on a protein surface according to claim 12, further comprising a processor, wherein the processor loads and executes the at least one instruction stored in the memory to implement the method for protein-protein docking based on identification of a low-entropy hydration layer on a protein surface.
